(19) United States
(12) Patent Application Publication
(10) Pub. No.: US 2026/0141982 A1
(43) Pub. Date: May 21, 2026

(54) RETRIEVAL METHOD, DATA RETRIEVAL APPARATUS, ELECTRONIC DEVICE, AND STORAGE MEDIUM

(71) Applicant: TENCENT TECHNOLOGY (SHENZHEN) COMPANY LIMITED, Shenzhen (CN)

(72) Inventors: Yu RONG, Shenzhen (CN); Liming WU, Shenzhen (CN); Fandi WU, Shenzhen (CN); Wenbing HUANG, Shenzhen (CN)

(21) Appl. No.: 19/450,491

(22) Filed: Jan. 15, 2026

Related U.S. Application Data
(63) Continuation of application No. PCT/CN2024/123972, filed on Oct. 10, 2024.

(30) Foreign Application Priority Data
Oct. 24, 2023 (CN) 202311387363.0

(51) Int. Cl. G16B 40/00; G06N 3/042

(52) U.S. Cl. CPC G16B 40/00; G06N 3/042

(57) ABSTRACT

A retrieval method includes: obtaining graph data corresponding to an input object, the graph data including a plurality of nodes and node data corresponding to each node; encoding and discretizing the graph data by using a graph encoder, to obtain a data sequence corresponding to the input object, the data sequence representing discretized information of at least one of node features or position features of the plurality of nodes, and the discretized information being obtained through the encoding; and retrieving, in a graph database, a target data sequence satisfying a similarity threshold with the data sequence, and determining an output object corresponding to the input object based on the retrieved target data sequence.

### CROSS-REFERENCES TO RELATED APPLICATIONS

This application is a continuation of PCT Application No. PCT/CN2024/123972, filed on Oct. 10, 2024, which claims priority to Chinese Patent Application No. 202311387363.0, filed on Oct. 24, 2023, the entire contents of all of which are incorporated herein by reference.

### FIELD OF THE TECHNOLOGY

The present disclosure relates to the field of artificial intelligence services and the cloud field, and more specifically, to a retrieval method, a data retrieval apparatus, an electronic device, a computer-readable storage medium, and a program product.

### BACKGROUND OF THE DISCLOSURE

With the rapid development of artificial intelligence technologies, artificial intelligence has shown great application prospects and development potential in the field of drug research and development. An artificial intelligence algorithm based on deep learning can efficiently analyze massive life science data, to implement accurate modeling and prediction on protein sequences and protein structures. This provides strong technical support for drug discovery. For example, during drug screening, artificial intelligence may rapidly compare tens of thousands of compound libraries, to find potential drug molecules that bind more closely to disease-related targets. This greatly shortens a research and development cycle of new drugs. In addition, artificial intelligence may further be configured for analyzing virus protein mutations, and predicting impact of the mutations on drug efficacy, and drugs that are equally effective against a new virus strain are developed based on this. Application space of artificial intelligence in new drug research and development continues to expand in the future, and intelligent assistance in entire procedures such as drug development, efficacy prediction, and formulation of a clinic test is implemented.

In current artificial intelligence-based research and development of new drugs, there are still some technical bottlenecks to be broken through. First, effective candidate compounds cannot be effectively screened out due to low prediction accuracy of an existing artificial intelligence-based drug discovery model. In addition, most of artificial intelligence-based drug discovery models have complex structures, and need to be supported by a large quantity of computing resources in an operation process, resulting in slow training and prediction speeds and excessively low efficiency. Moreover, there is still a lack of an integrated online drug discovery platform currently, making it difficult for research and development personnel to directly search for drugs (especially a drug with a complex protein structure) online and obtain a visual result. Consequently, inconvenience is caused to the research and development personnel.

Therefore, a related technology needs to be further improved.

### SUMMARY

Embodiments of the present disclosure provide a retrieval method, a data retrieval apparatus, an electronic device, and a computer-readable storage medium.

An embodiment of the present disclosure provides a retrieval method, used to retrieve an input object in a graph database. The retrieval method includes: obtaining graph data corresponding to the input object, the graph data including a plurality of nodes and node data corresponding to each node; encoding and discretizing the graph data by using a graph encoder, to obtain a data sequence corresponding to the input object, the data sequence representing discretized information of at least one of node features or position features of the plurality of nodes, and the discretized information being obtained through the encoding; and retrieving, in the graph database, a target data sequence satisfying a similarity threshold with the data sequence, and determining an output object corresponding to the input object based on the retrieved target data sequence.

An embodiment of the present disclosure provides a data retrieval apparatus, configured to retrieve an input object in a graph database. The apparatus includes: a graph data constructing module, configured to obtain graph data corresponding to the input object, the graph data including a plurality of nodes and node data corresponding to each node; an encoding module, configured to encode and discretize the graph data by using a graph encoder, to obtain a data sequence corresponding to the input object, the data sequence representing discretized information of at least one of node features or position features of the plurality of nodes, and the discretized information being obtained through encoding; and a comparison module, configured to: retrieve, in the graph database, a target data sequence satisfying a similarity threshold with the data sequence, and determine an output object corresponding to the input object based on the retrieved target data sequence.

An embodiment of the present disclosure discloses an electronic device, including one or more processors; and one or more memories, the memory having a computer-executable program stored therein, and the processor performing the foregoing method when executing the computer-executable program.

An embodiment of the present disclosure provides a non-transitory computer-readable storage medium, the computer-readable storage medium having computer instructions stored therein, and the foregoing method being performed when the computer instructions are executed by a processor.

In the embodiments of the present disclosure, the graph encoder is used to encode graph data associated with an input object having a three-dimensional space structure, and a sufficiently accurate data sequence is generated, so that an output object similar to the input object in terms of a three-dimensional structure and/or a node feature can be accurately detected in the graph database.

In particular, in the field of life science data analysis, according to the embodiments of the present disclosure, an equivariant vector quantized variational encoder is used to encode a protein structure, to generate a sufficiently accurate sequence configured for representing a complex protein structure, so as to accurately retrieve a candidate protein that is most similar to an inputted protein in structure. In addition, in some embodiments of the present disclosure, protein retrieval may be performed based on sequence comparison, to improve retrieval efficiency. In addition, some embodiments of the present disclosure further provide a solution to train a retrieval model. In the embodiments of the present disclosure, the retrieval model is trained at least partially based on an amino acid prediction loss and a coordinate alignment loss, to obtain a retrieval model with higher precision and better performance.

### BRIEF DESCRIPTION OF THE DRAWINGS

To describe technical solutions in embodiments of the present closure more clearly, accompanying drawings required for describing the embodiments are briefly described below. The accompanying drawings in the following descriptions are merely exemplary embodiments of the present disclosure.

FIG. 1 is a diagram of an example of a scenario according to an embodiment of the present disclosure;

FIG. 2 is a diagram of an application scenario according to an embodiment of the present disclosure;

FIG. 3 is a flowchart of a graph data processing method according to an embodiment of the present disclosure;

FIG. 4 is a diagram of graph data according to an embodiment of the present disclosure;

FIG. 5 is a diagram of a graph encoder according to an embodiment of the present disclosure;

FIG. 6 is a diagram of operation S303 according to an embodiment of the present disclosure;

FIG. 7 is a diagram of training a graph encoder according to an embodiment of the present disclosure;

FIG. 8 is a diagram of an electronic device according to an embodiment of the present disclosure;

FIG. 9 is a diagram of an architecture of an example of a communication device according to an embodiment of the present disclosure; and

FIG. 10 is a diagram of a storage medium according to an embodiment of the present disclosure.

### DESCRIPTION OF EMBODIMENTS

To make objectives, technical solutions, and advantages of the present disclosure more clear, the following describes in detail exemplary embodiments of the present disclosure with reference to the accompanying drawings. Clearly, the described embodiments are merely some but not all of the embodiments of the present disclosure. The present disclosure is not limited by the exemplary embodiments described herein.

In the specification and the accompanying drawings, operations and elements that are basically the same or similar are represented by the same or similar reference signs, and repeated descriptions of these operations and elements are omitted. Meanwhile, in descriptions of the present disclosure, terms such as “first” and “second” are merely used for distinguishing between descriptions, and cannot be understood as indicating or implying relative importance or a sequence.

For ease of describing the present disclosure, concepts related to the present disclosure are introduced below.

Protein engineering is an important frontier technology that aims to properly construct an entirely new protein molecule from the very beginning as required, to enable the protein molecule to have new activities, thereby implementing a new function or another specific objective. The protein engineering may be completely from the very beginning. An entirely new amino acid sequence is created on a computer according to a biophysical principle, and a three-dimensional structure thereof is predicted. The protein engineering may alternatively be carried out by introducing an amino acid substitution into a known natural protein, to generate a variant with new properties. The present disclosure is applicable to the foregoing two protein engineering solutions. For example, in the present disclosure, a corresponding amino acid sequence may be predicted for a specific protein structure. The amino acid sequence facilitates subsequent protein construction.

A graph neural network (GNN) is a machine learning model configured to process graph data. Different from a conventional neural network model, the GNN may process graph data of any shape and size, for example, a social network, a chemical molecule, or a transportation network. A main idea of the GNN is to convert the graph data into a vector or matrix form, and then perform training and prediction by using a neural network. A main advantage of the GNN is that the GNN can process the graph data of any shape and size, and has strong flexibility and adaptability. In addition, the GNN can extract key information and features from the graph data, to better understand and process the graph data. The GNN is very widely applied. For example, in the social network, the GNN may be configured to predict interests and behaviors of a user; in the chemical molecule, the GNN may be configured to predict a property and a response of the molecule; and in the transportation network, the GNN may be configured to predict a traffic flow and a congestion status. An equivariant graph neural network is a special GNN that incorporates geometric features of a junction point, for example, three-dimensional features such as coordinates and a velocity. The equivariant graph neural network is very common in the field of AI4Science. An output of the equivariant graph neural network changes regularly as an input changes, and the change generally satisfies SE(3).

Dynamic programming (DP) is an algorithm configured for resolving a multi-stage decision problem. The DP is widely applied to resolve multi-stage optimization decision problems, such as a knapsack problem and a shortest path. In the DP, a global optimal solution is deduced by using an optimal solution of a sub-problem, avoiding repeated calculation and achieving high calculation efficiency. In the dynamic programming algorithm, a complex problem is decomposed into a plurality of cross-correlation sub-problems or decision stages, and a mathematics model is established for each sub-problem to describe a state transfer relationship thereof, that is, an optimal relationship between the sub-problems. In an embodiment, in a process of DP, the most basic or simplest sub-problem may be first resolved, and then an optimal solution at each stage is solved sequentially based on a state transfer equation in each problem by using a step-by-step recursion method from bottom to top, to finally obtain a global optimal solution of the problem. For example, in an example, the dynamic programming algorithm usually includes the following operations. Division stage: Divide a problem into several stages, and take a different decision at each stage. Determine a state: Determine a state at each stage, the state usually being represented by using a state variable. State transfer equation: Determine a state transfer relationship between different stages, the state transfer relationship usually being represented by using a recursive formula. Boundary condition: Determine a boundary condition of a problem, that is, a solution of a minimum sub-problem. Solving objective: Calculate an optimal solution of the problem based on the state transfer equation and the boundary condition. In the dynamic programming algorithm, calculation is usually performed in a bottom-to-top manner, that is, step-by-step recursion is performed from the minimum sub-problem until the optimal solution of the entire problem is solved. In a calculation process, a two-dimensional array usually needs to be used to store states at different stages, to facilitate state transfer.

A retrieval model in the present disclosure may be based on artificial intelligence (AI). For example, the retrieval model in the present disclosure can retrieve information about a protein in a manner similar to how humans understand a spatial feature of a protein structure, to study the protein structure and a protein function, so as to infer a biological property of the protein. AI can study construction principles and implementation methods of various intelligent machines, to enable the retrieval model in the present disclosure to have a capability of understanding the protein.

The embodiments of the present disclosure mainly relate to a graph learning direction in machine learning and deep learning, and are usually applied to actual problems such as a social network, e-commerce recommendation, modeling of drug molecules, and cloud.

In some embodiments, a computer vision technology used in the embodiments of the present disclosure may further be based on machine learning (ML) and deep learning. ML and deep learning usually include technologies such as an artificial neural network, a belief network, reinforcement learning, transfer learning, and inductive learning.

In some embodiments, all the following retrieval models that can be used in the embodiments of the present disclosure may be artificial intelligence models, and in particular, artificial intelligence-based neural network models. Usually, the artificial intelligence-based neural network model is implemented as an no-ring graph, in which neurons are arranged on different layers. Usually, the neural network model includes an input layer and an output layer, and the input layer and the output layer are separated by using at least one hidden layer. The hidden layer transforms an input received by the input layer into a representation representing that an output generated by the output layer is useful. Network nodes are all connected to a node on an adjacent layer by using edges, and there is no edge between nodes on each layer. Data received at a node on the input layer of the neural network is propagated to a node on the output layer through any one of the hidden layer, an activation layer, a pooling layer, a convolutional layer, and the like. The input and the output of the neural network model may use various forms. This is not limited in the present disclosure.

The solutions provided in the embodiments of the present disclosure relate to technologies such as AI, deep graph learning, or ML, and are specifically described by using the following embodiments.

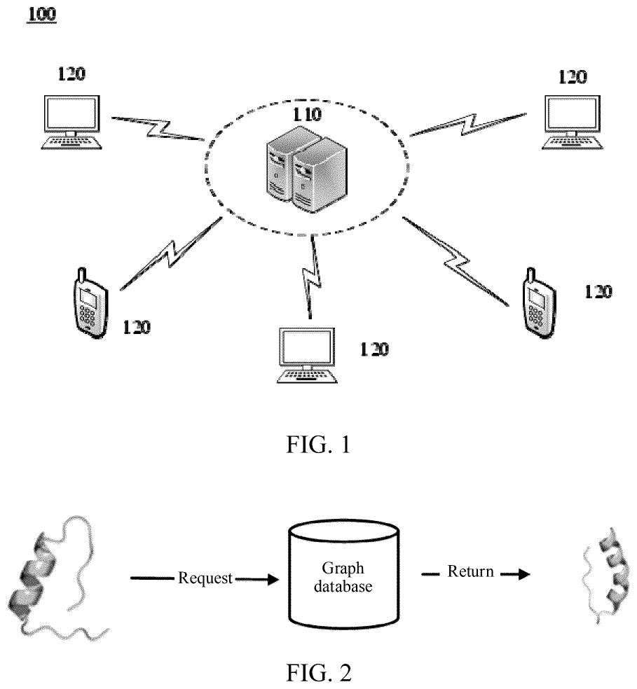

First, an application scenario of a graph data processing method, a corresponding apparatus, and the like according to embodiments of the present disclosure is described with reference to FIG. 1. FIG. 1 is a diagram of an application scenario 100 according to an embodiment of the present disclosure. A server 110 and a plurality of terminals 120 are schematically shown.

A retrieval model in the embodiments of the present disclosure may be specifically integrated into various electronic devices, for example, any electronic device of the server 110 and the plurality of terminals 120 in FIG. 1. For example, the retrieval model may be integrated into the terminal 120. The terminal 120 may be a smartphone, a tablet computer, a notebook computer, a desktop computer, a personal computer (PC), a smart speaker, a smart watch, or the like, but is not limited thereto. For another example, the retrieval model may alternatively be integrated into the server 110. The server 110 may be an independent physical server, may be a server cluster or a distributed system that includes a plurality of physical servers, or may be a cloud server that provides basic cloud computing services such as a cloud service, a cloud database, cloud computing, a cloud function, cloud storage, a network service, cloud communication, a middleware service, a domain name service, a security service, a content delivery network (CDN), big data, and an artificial intelligence platform. The terminal and the server may be connected directly or indirectly in a wired or wireless communication manner. This is not limited in the present disclosure.

An apparatus that performs inferring by using the retrieval model in the embodiments of the present disclosure may be a terminal, may be a server, or may be a system including a terminal and a server. The graph data processing method in the embodiments of the present disclosure may be performed by the terminal, may be performed by the server, or may be performed by the terminal and the server together.

The retrieval model provided in the embodiments of the present disclosure may further relate to an artificial intelligence cloud service in the field of cloud technologies.

The terminal 110 and the server 120 according to this embodiment of the present disclosure both comply with a data protection principle, respect data rights of a user, and ensure data security and privacy of the user. The terminal 110 and the server 120 according to this embodiment of the present disclosure explicitly inform the user of a purpose, a manner, and a scope of collecting, using, storing, transmitting, and deleting data of the user, and obtain consent of the user. The terminal 110 and the server 120 according to this embodiment of the present disclosure use proper technologies and management measures to prevent the data of the user from being leaked, tampered, damaged, or lost. A provider of the terminal 110 and the server 120 according to this embodiment of the present disclosure regularly reviews and updates the data of a user, and delete expired or useless data in time. In addition, a cloud service provider using this embodiment of the present disclosure respect rights such as data access, correction, deletion, and withdrawal of consent, complaints, and claims of the user, and provides a convenient channel and a program, so that the user can effectively exercise these rights.

Further, a process of data analysis of a drug (in particular, a protein) by using the retrieval model in the terminal 120 or the server 110 is performed based on a legal, proper, and transparent principle. Data collected and processed by the retrieval model according to the embodiments of the present disclosure is relevant, necessary, and appropriate for a prediction purpose, and does not include any personal identity information or sensitive information. The retrieval model according to the embodiments of the present disclosure uses appropriate technologies and organization measures, to protect security and integrity of the data, and prevent the data from unauthorized access, use, or leakage.

The artificial intelligence-based retrieval model according to the embodiments of the present disclosure complies with related data protection regulations and ethical principles. The retrieval model is trained based on a large amount of anonymized and de-identified data, and does not violate private rights and intellectual property of any individual or group. The retrieval model is also strictly tested and evaluated, to ensure that an output result of the retrieval model is accurate and reliable, and does not cause any misleading or ambiguity. The retrieval model is developed only for improving service quality and customer satisfaction, and is not used for any illegal or unscrupulous purpose. In addition, the retrieval model is reviewed and updated periodically, to adapt to changes of a data environment and legal norms.

A retrieval model in a related technology usually uses one or more of the following methods to determine a similarity between proteins: an amino acid sequence/order-based method, a protein structure-based method (for example, a structure-based full-atomic model method, a main framework atom-based method, and a structural segment-based method), a fuzzy matching-based method, and the like. The methods have respective advantages and disadvantages. For example, although a calculation speed of the amino acid sequence-based method is high, a similarity calculated in the method is usually not accurate enough because a space structure of the protein is not considered in the method. The structure-based calculation method has high calculation costs, a low speed, and excessively low efficiency.

Moreover, in the related technology, for a situation in which the same protein is in different views, it is difficult to parse and calibrate the protein in different views in most methods. Consequently, once a view of observing the protein changes, a retrieval result clearly deviates. In addition, because the related technology has disadvantages such as inaccurate retrieval results and low retrieval efficiency in various aspects of the protein, it is difficult to construct a system-complete online search and visualization platform, mine and apply massive life science data, and directly provide real-time online services for biologists, causing inconvenience to subsequent research.

In the embodiments of the present disclosure, a graph encoder is used to encode graph data associated with an input object having a three-dimensional space structure, and a sufficiently accurate data sequence is generated, so that an output object corresponding to the input object (e.g., similar to the input object) in terms of a three-dimensional structure and/or a node feature can be accurately detected in a graph database. For example, in an implementation, a complex space structure of the input object may be first discretely encoded into a sequence by using an equivariant graph neural network and a vector quantized variational autoencoder (VQ-VAE), and then a similarity between “structure sequences” of different objects is calculated by using a sequence comparison algorithm, to return a candidate input object most similar to the searched input object in structure.

Therefore, especially in the field of life science data analysis, according to the embodiments of the present disclosure, based on AI, an equivariant VQ-VAE is used to encode the protein structure, to generate a sufficiently accurate sequence configured for representing a complex protein structure, so as to accurately retrieve a candidate protein that is most similar to inputted protein in structure, for example, a candidate protein that satisfies a similarity threshold in structure. In the embodiments of the present disclosure, because a retrieval speed can be greatly improved, real-time online services can be directly provided for biologists, bringing great convenience to subsequent research.

In addition, in some embodiments of the present disclosure, protein retrieval may be performed based on sequence comparison, to improve retrieval efficiency. Moreover, some embodiments of the present disclosure further provide a solution to train the retrieval model. In the embodiments of the present disclosure, the retrieval model is trained at least partially based on an amino acid prediction loss and a coordinate alignment loss, to obtain a retrieval model with higher precision and better performance.

The following describes, with reference to FIG. 2 to FIG. 10, a graph data processing method according to an embodiment of the present disclosure.

FIG. 2 is a diagram of an application scenario according to an embodiment of the present disclosure.

As shown in FIG. 2, this embodiment of the present disclosure may be integrated into a protein database search tool based on deep learning. The protein database search tool mainly includes an input module, a database module, a structure retrieval module, a result visualization module, and an algorithm model module. The input module inputs a protein having a three-dimensional structure (also referred to as inputted protein below) shown in FIG. 2, and the input module parses the three-dimensional structure of the inputted protein, and generates a retrieval request based on a parsing result. The input module sends the retrieval request to the database module and the structure retrieval module, to find one or more proteins having a structure similar to that of the inputted protein in a protein structure database.

This embodiment of the present disclosure facilitates study a new protein. For example, the structure and a function of the inputted protein may be studied and a biological property of the inputted protein may be predicted by searching the protein database for the protein having the structure similar to that of the inputted protein. Therefore, the protein database search tool integrated with this embodiment of the present disclosure may be configured to analyze biological big data, to discover new biological rules and properties. In addition, the protein database search tool may further be configured to analyze structure information of a known protein in the protein database, to find or generate a new protein structure, so as to implement a specific function.

Certainly, this embodiment of the present disclosure may further be integrated into another biological data analysis tool, to provide support for biomedical research in various fields. According to this embodiment of the present disclosure, a protein candidate having a structure highly similar to that of the inputted protein may be quickly searched for with high precision in the protein database based on the three-dimensional structure of the inputted protein. Because the embodiments of the present disclosure have advantages such as a high search speed and high search precision, the embodiments of the present disclosure have wide application scenarios, including but not limited to: (1) predicting the structure and the function of the protein by comparing/querying homologies between the inputted protein and a known protein in the database; (2) assisting in, based on a query result and the structure information in the database in a process of drug research and development, identification of an amino acid that may be used as a new target; (3) assisting in, based on a retrieval result and the structure information in the database, identification of an amino acid that may mutate, and determining impact on the function of the protein based on the identification; and (4) using protein structure data as a dimension of analyzing and processing life science big data, to provide support for subsequent analysis of the life science big data. A person skilled in the art is to understand that the present disclosure is not limited thereto.

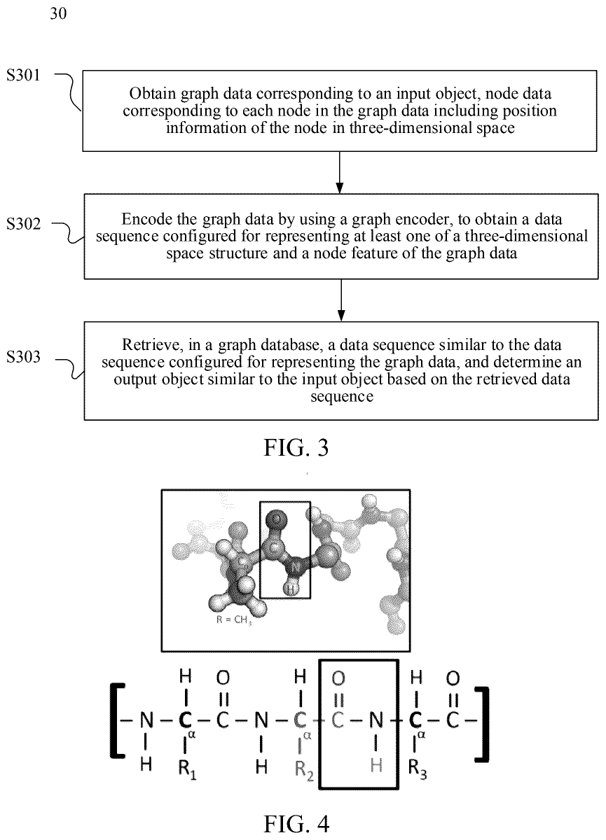

FIG. 3 is a flowchart of a graph data processing method 30 according to an embodiment of the present disclosure.

The method 30 may be performed by a terminal device (for example, the terminal 120 shown in FIG. 1). The method 30 includes operation S301 to operation S303. Certainly, the method 30 may further include more or fewer operations. This is not limited in the present disclosure.

The method 30 is used to retrieve an input object in a graph database. The graph database is a database providing management and analysis capabilities of graph structural data. The graph database can manage complex graph data having an association relationship, and can provide an implicit connection analytic function such as pattern matching. The graph database can support query and retrieval for the graph data. Alternatively, the graph database may even be accessed in a chain manner along with a node relationship in the input graph data. In comparison with a relational database, the graph database is better at managing a dependency relationship between data objects, and has wide application. Certainly, the present disclosure is not limited thereto.

Operation S301: Obtain graph data corresponding to the input object, the graph data including a plurality of nodes (also referred to as a junction point in some applications) and node data corresponding to each node. The node data corresponding to each node may include position information of the node in three-dimensional space, for example, actual coordinates of the node in the three-dimensional space. Further, the node data corresponding to each node may include identification information, for example, type information, configured for identifying each node.

In an embodiment, the input object is an object, a system, or a data structure having a three-dimensional feature, and can be described and analyzed in the three-dimensional space. Specifically, the input object may have a plurality of internal components. The internal components are individual elements, parts, or sections forming the input object. These internal components may be independent entities, or may be mutually dependent to form a larger whole together. In addition, these internal components may further be arranged or distributed in three dimensions in the three-dimensional space. For example, the input object may be a protein or a macromolecule, and the internal components of the input object may be amino acids or internal atoms of the amino acids forming the protein or the macromolecule. These amino acids and internal atoms of the amino acids may interact with each other, and be distributed in the three-dimensional space, to provide special biological properties of the protein/macromolecule. For another example, the input object may be a complex social network, and an internal component of the social network may be one or more persons participating in social interactions. These persons participating in the social interactions may have corresponding geographical location coordinates in the three-dimensional space. However, two persons whose geographical locations are closer may be easier to be friends. Certainly, the present disclosure is not limited thereto.

The graph data according to this embodiment of the present disclosure is data that can present an association relationship between nodes in a data structure form of a “graph”. The “graph” is, for example, an undirected graph. The undirected graph includes a limited (which may be variable) set as a node set, and an undirected pair (corresponding to the undirected graph) set as an edge set. In addition, the “graph” may alternatively be a directed graph. This is not limited in the present disclosure. The node is also referred to as a vertex, and is a basic unit forming a data structure such as the “graph”. Each node may correspondingly have node data. The node data may be a part of data associated with the node in the graph data, or may be an external entity represented by using an integer subscript or a reference. In addition to the node data, a data structure of the graph data may further include edges, and each edge may be correspondingly associated with a value (edge value), for example, a label or a value (that is, a weight). Therefore, mathematically, the graph data according to this embodiment of the present disclosure may be described as a set of nodes and edges, that is, G=<V, E>. Each edge e=(u, v)∈E connect two nodes u and v, and may represent a relationship between the nodes.

For the graph data corresponding to the input object, each node in the graph data corresponds to one or more internal components in the input object. The node data corresponding to each node may include identification data configured for identifying one or more internal components in the input object. Each piece of edge data represents an association relationship between any two internal components in the input object. Because the input object has a three-dimensional space structure, to capture a relative position relationship between the internal components in the input object in the three-dimensional space, the node data corresponding to each node in the graph data may include the position information of the node in the three-dimensional space. The position information of the node in the three-dimensional space may be represented by using three-dimensional coordinates (x, y, z). Certainly, the present disclosure is not limited thereto.

In an embodiment, the graph data satisfies at least one of rotation equivariance, translation equivariance, and scaling equivariance. Graph data that satisfies all the three conditions is also referred to as satisfying SE(3) invariance. In other words, even if operations such as translation, rotation, and scaling are performed on the graph data in the three-dimensional space, an output does not change. For example, even if node data corresponding to each node in the graph data is constructed for different three-dimensional space, a relative position between nodes does not change. In addition, even if the three-dimensional space changes (for example, different three-dimensional coordinate systems are created), a sequence outputted based on the graph data remains unchanged. A detailed process of obtaining the graph data corresponding to the input object is described below with reference to FIG. 4, and details are not described herein again in the present disclosure.

Operation S302: Encode and discretize the graph data by using a graph encoder, to obtain a data sequence corresponding to the input object, the data sequence representing discretized information of at least one of node features or position features of the plurality of nodes, and the discretized information being obtained through encoding. The node feature of a node may include node feature information such as a type of the node and an association relationship between the node and another node, and the position feature of a node may include position information obtained through encoding that is of the node and that is different from actual coordinates of the node in the graph data but corresponds to the actual coordinates. It can be learned that the data sequence can reflect information such as a structure of the input object.

In an embodiment, the data sequence may be generated by the graph encoder in the foregoing retrieval model. The retrieval model may be any neural network model using a graph representation learning technology. The graph encoder in the retrieval model may learn of, from the graph data, a vector of a feature representation (that is, the node feature and the position feature of each node that are encoded) corresponding to the graph data or a vector of a node feature corresponding to any node in the graph data, and perform discretization processing such as clustering on a vector of the node feature and/or a vector of the position feature, to obtain the data sequence representing the discretized information the node features and/or the position features of the plurality of nodes in the graph data. In an example, the data sequence may include a cluster center of the node features of the plurality of nodes.

In an embodiment, the graph encoder is a neural network that can be configured to encode the graph data into an embedding or a vector. The graph encoder updates an embedding representation of each node by using propagation information along an edge in the graph data, to learn of the relationship between the nodes, and further respectively generate vectors of the node features and vectors of the position features of the nodes. Then, the vector of the node feature and/or the vector of the position feature may be converted into the data sequence by using one or more neural networks, perceptrons, or quantizers. The data sequence is a set of data elements arranged in a specific order. The data element may be a value, a character, a vector, or the like. In addition, these elements are stored in a specific order. A data element in each position may be accessed by using an index. For example, the data sequence may be an array having a fixed length. For a protein input object, the data sequence may be a cluster center of a node feature of each node. Certainly, the present disclosure is not limited thereto.

In an embodiment, the graph data obtained in operation S301 satisfies SE(3) invariance (or SE(3) equivariance). Therefore, a graph encoder that can encode such graph data may be an equivariant graph neural network (EGNN) model. An EGNN is a neural network architecture configured to process the graph data, and a key idea of the EGNN is to maintain equivariance when the graph data is processed. It means that transformation (for example, rotation, translation, or scaling) performed by the EGNN on the input data does not change the output. Therefore, such a graph encoder can more effectively adapt to the graph data having the three-dimensional space structure. Certainly, the present disclosure is not limited thereto.

In an embodiment, the graph encoder may alternatively be a vector quantized variational autoencoder (VQ-VAE), and is a deep learning model. The VQ-VAE combines ideas of an autoencoder and vector quantization, and is configured to learn of a compressed representation of high-dimensional data. The VQ-VAE can map the inputted graph data to latent representation space. In a training process, a vector quantized variational decoder is usually correspondingly determined to restore the latent representation to original graph data. If a difference between graph data obtained through restoration and the original graph data is small enough, it indicates that training is completed. In comparison with another autoencoder, a latent representation generated by the VQ-VAE is quantized through vector quantization. Specifically, the latent representation space is divided into a group of pieces of discrete subspace, and the latent representation generated by the VQ-VAE is mapped to each piece of subspace, to generate a group of sequences. In this way, dimensions are reduced and robustness is increased.

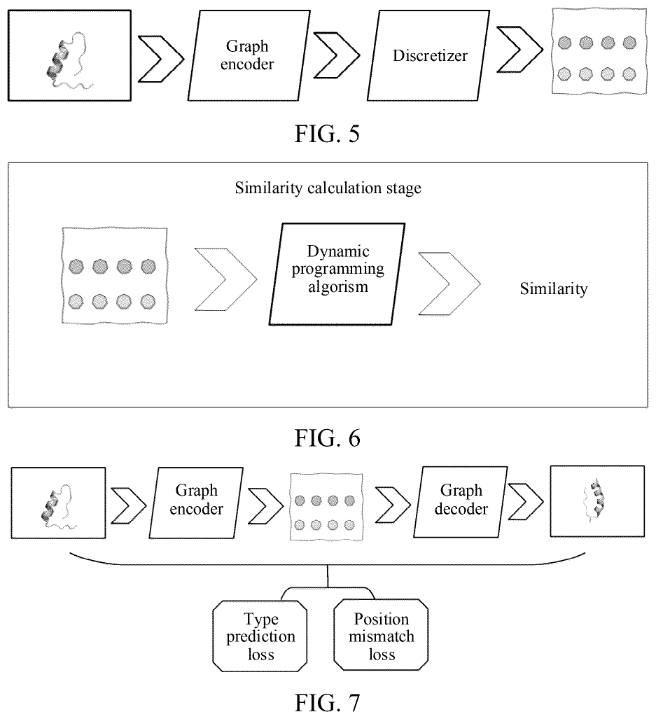

In an embodiment, the graph encoder may alternatively be an equivariant VQ-VAE combining the EGNN and the VQ-VAE. In other words, the graph encoder can obtain the same vector quantization sequence by performing operations such as rotation, translation, and scaling on the graph data. The equivariant VQ-VAE is used, so that when SE(3) equivariance of the graph data is effectively used, the dimensions can be reduced and the robustness can be increased. The equivariant VQ-VAE according to this embodiment of the present disclosure is further described below with reference to FIG. 5. Certainly, the present disclosure is not limited thereto.

Operation S303: Retrieve, in the graph database, a target data sequence satisfying a similarity threshold with the data sequence of the graph data, and determine an output object corresponding to the input object based on the retrieved target data sequence. The output object has similar three-dimensional structure and/or node features as the input object.

In an embodiment, the data sequence similar to the data sequence configured for representing the graph data may be determined based on a similarity between two data sequences. The similarity between the data sequences can be configured for measuring a degree of a difference between two data sequences. In an embodiment, the similarity between the data sequences may be calculated by using one or more of the following methods: a Hamming distance, a cosine similarity, an elastic distance, a structure difference, a linear correlation, a longest common sub-sequence editing distance, and the like. Certainly, the present disclosure is not limited thereto.

In an embodiment, the data sequence similar to the data sequence obtained in operation S302 may be retrieved in the graph database by using a dynamic programming-based sequence comparison algorithm (for example, a Smith-Waterman algorithm). If a similarity between a data sequence corresponding to the input object and a data sequence corresponding to an object in the graph database is high, it may be considered that a similarity between the two objects is high. Therefore, the object may be used as the output object. Some details of operation S303 are further described below with reference to FIG. 6. Details are not described in the present disclosure again.

Therefore, according to the embodiments of the present disclosure, the graph encoder is used to encode and discretize graph data associated with the input object having the three-dimensional space structure, and a sufficiently accurate data sequence is generated, so that the output object corresponding to the input object in terms of a three-dimensional structure and/or a node feature can be accurately detected in the graph database. In comparison with a related technology, according to the embodiments of the present disclosure, not only retrieval precision is high, but also retrieval efficiency is significantly improved.

Specifically, in the field of protein/macromolecule retrieval, according to the embodiments of the present disclosure, in some embodiments, the graph encoder (especially an equivariant vector quantized variational encoder) may be used to encode and discretize a protein structure, to generate a sufficiently accurate data sequence configured for representing a complex protein structure, so as to accurately retrieve a candidate protein that is most similar to the inputted protein in structure. In addition, in some embodiments of the present disclosure, protein retrieval may be performed based on sequence comparison, to improve the retrieval efficiency.

Next, operation S301 is further described with reference to FIG. 4. FIG. 4 is a diagram of graph data according to an embodiment of the present disclosure. An input object corresponding to the graph data is a protein/macromolecule.

As shown in FIG. 4, the protein may be represented in a form of the graph data shown in FIG. 4. In operation S301, graph data corresponding to the inputted protein is obtained. As described above, the graph data includes node data and edge data. Operation S301 includes: Determine, based on sequence information of a plurality of amino acids included in the protein, each node and node data of the node in the graph data corresponding to the input object, each node corresponding to one amino acid, the node data corresponding to each node including type information of atoms on a main chain of the amino acid and coordinates of the atoms in three-dimensional space; and determine each piece of edge data in the graph data based on the node data of each node in the graph data. Certainly, the present disclosure is not limited thereto.

In an embodiment, a process of obtaining the graph data corresponding to the inputted protein may be briefly described as follows. When representing a protein, protein data is usually stored in a protein data bank (PDB) file, and the file includes amino acid sequence information of the protein, and coordinate information of the protein in the three-dimensional space. A type of each amino acid (or amino acid residue) on a protein fragment and three-dimensional coordinates of four atoms (C, N, O, Cₐ) on a main chain thereof are considered. In an embodiment, in the graph data, each node may correspond to an amino acid (or an amino acid residue) on the protein fragment, and node data corresponding to each node may include and is not limited to: coordinates of the node, to be specific, types of four atoms on a main chain of the amino acid (or the amino acid residue) and three-dimensional coordinates corresponding to each atom. For example, a protein sequence having n amino acids may be represented by using an amino acid sequence A={a₁, a₂ . . . aₙ} and coordinate information X={x₁, x₂, x₃, . . . , xₙ}∈ [symbol] , xᵢ∈R^{4×3} representing three-dimensional coordinates of four atoms on a main chain of an i^{th} amino acid.

A visual example of xᵢ is shown in a three-dimensional figure circled by a black box in the upper part of FIG. 4, and corresponds to an amino acid fragment circled by a black box in the lower part of FIG. 4. Although in the present disclosure, corresponding node data is constructed by using three-dimensional coordinates of the amino acid as structure information, the present disclosure is not limited thereto. For example, in the present disclosure, other data may further be used to extend data that is in the node data and that represents a structure feature of the protein, for example, a dihedral angle between amino acids and a biological attribute of the amino acid.

In an embodiment, the edge data may be determined based on the foregoing obtained node data. The determining each piece of edge data in the graph data based on the node data of each node in the graph data includes: creating and storing edge data corresponding to a chemical bond based on the chemical bond between atoms of two amino acids; or creating and storing, in response to that a distance between atoms of two amino acids is less than a predetermined threshold, edge data for connecting the atoms of the two amino acids.

Specifically, a process of obtaining the edge data may be briefly described as follows.

In some embodiments of the present disclosure, each piece of edge data may be configured for representing one chemical bond. If it can be determined, based on node data of two amino acids, that one chemical bond exists between atoms of the two amino acids, edge data corresponding to the chemical bond is correspondingly created and stored in the graph data. In an embodiment, the edge data further includes feature data, the feature data indicating a feature of the chemical bond, for example, a single bond or a double bond. Certainly, the present disclosure is not limited thereto.

In some other exemplary embodiments of the present disclosure, each piece of edge data may be configured for representing distance information between atoms of two amino acids. For example, if it can be determined, based on node data of two amino acids, that a distance between atoms of the two amino acids is less than a predetermined threshold, a piece of edge data may be correspondingly created and stored in the graph data. Usually, strong interactions in the macromolecule or protein exist between atoms that are close to each other. However, during subsequent data analysis, interactions between atoms facilitate information transfer. Therefore, the edge data created based on a distance between atoms facilitates analysis of a macromolecule or protein structure. Specifically, in the embodiments, the edge data may be created in the following manner. First, a distance threshold td is determined (for example, the distance threshold td is inputted into an input module). A distance between atoms Cₐ of any two amino acids in the inputted protein is calculated. If the distance is less than the threshold td, one edge may be connected between the two amino acids (in other words, one piece of edge data is stored).

In this way, a graph representation (that is, the graph data) G=(V,E,X) of the protein can be obtained, V={v₁, v₂ . . . vₙ} representing n nodes, and each node may correspond to one amino acid. E={(vᵢ, vⱼ)|dist{vᵢ, vⱼ}<td} represents an edge set in the graph, X={x₁, x₂ . . . xₙ)} represents coordinates of the node, and xᵢ represents three-dimensional coordinates of a node vᵢ. When one node corresponds to one amino acid, and a main chain of one amino acid includes four atoms, xᵢ∈R^{4×3} represents coordinates of four atoms on a main chain of an i^{th} amino acid.

In a representation solution, a representation of the node and the coordinates of the node are separately stored in an index manner. This helps subsequent calculation of an equivariant vector quantized variational encoder. Certainly, the present disclosure is not limited thereto.

Alternatively, graph data G=(X,E) may be used to represent a protein, X={x₁, x₂ . . . xₙ} representing coordinates of a node, and xᵢ representing three-dimensional coordinates of a node having a node identifier i. E={(vᵢ, vⱼ)|dist{vᵢ, vⱼ}<td} represents an edge set in the graph. In a representation solution, a representation of the node and the coordinates of the node are separately stored in an index manner. This helps reduce storage space. Certainly, the present disclosure is not limited thereto.

Although FIG. 4 only shows the graph data representing the protein or macromolecule, a person skilled in the art can understand that this embodiment of the present disclosure is not limited to only processing the protein or macromolecule, and a solution thereof may also be similarly applied to other fields such as a social network, an event graph, a knowledge graph, and an e-commerce platform. In these fields, the node data and the edge data correspondingly have physical meanings thereof. For example, in a social network analysis scenario, the node data may represent users, and the edge data represents a social relationship between the users; in the knowledge graph, the node data represents entities, and the edge data is a semantic relationship between the entities; in the e-commerce platform, a user, a commodity, a category, and the like may be abstracted as the node data in the graph, and behaviors such as purchasing and clicking/tapping may be abstracted as the edge data.

Although FIG. 4 only shows the graph data corresponding to the protein in a form of a mesh graph and a molecular formula structure, a person skilled in the art may additionally cite other information and integrate the information into the node data of each node. For example, the information may include information about a multi-level structure of the protein, to implement multi-level structure matching. In addition, the node data may further include sequence features such as a chemical property of the amino acid and a codon fragment, or information configured for representing the amino acid, such as a subunit relationship of a sub-cellular structure, to assist in parsing a three-dimensional structure of the graph data. In addition, even data in another modality, such as picture data (for example, a picture of an amino acid photographed under a microscope), may further be integrated into the node data. The data in another modality may be combined with the data sequence and/or structure information involved in this embodiment of the present disclosure, so that in this embodiment of the present disclosure, the protein structure can be retrieved in a multi-modality situation, to further improve retrieval precision.

This embodiment of the present disclosure actually provides a general graph data processing solution, and the solution can be compatible with various types of node data and edge data. In the embodiments of the present disclosure, a graph encoder accurately encodes the graph data into a sequence representation, and executes a subsequent retrieval and analysis task based on the sequence representation, to better serve requirements in various fields related to graph data processing.

Next, operation S302 is further described with reference to FIG. 5. FIG. 5 is a diagram of a graph encoder according to an embodiment of the present disclosure. The graph encoder is a sub-component of the foregoing retrieval model.

As shown in FIG. 5, the graph encoder may include an EGNN model and a discretizer. In an embodiment, in operation S302, the EGNN model and the discretizer in the graph encoder may cooperate to obtain the data sequence. Specifically, the encoding and discretizing the graph data by using a graph encoder includes: encoding the graph data by using the EGNN model, to obtain a node feature and a position feature of each node in the graph data; and performing vector quantization on at least one of the node feature or the position feature of each node by using the discretizer, to obtain a data sequence configured for representing discretized information of at least one of the node feature or the position feature of each node. Certainly, the present disclosure is not limited thereto.

The EGNN model is a multi-channel EGNN model. The EGNN includes a plurality of layers of perceptrons or hidden layers. An input of each hidden layer and/or perceptron is an output of a previous hidden layer and perceptron. To be specific, each hidden layer or perceptron of the EGNN encodes a vector of the node feature and a vector of the position feature of each node, and encoding of each hidden layer or perceptron other than the 1^{st} hidden layer or perceptron includes: obtaining vectors of node features and vectors of position features of a first node and a second node in the graph data, the vectors being encoded by a previous hidden layer or perceptron in the EGNN model; determining a propagation message of the second node for the first node based on the vector of the node feature and the vector of the position feature; and determining, based on the propagation message, a vector of a node feature and a vector of a position feature that are encoded by the hidden layer or perceptron.

Specifically, for the graph data in this embodiment of the present disclosure, an input of a l^{th} hidden layer/perceptron is a position feature, for example, node coordinates x^{l} and a node feature representation h^{l} that are outputted by a previous hidden layer/perceptron, and an output is node coordinates x^{l+1} and a node feature representation h^{l+1} changed based on the hidden layer or perceptron by using a message transfer mechanism. The process may be represented as a formula (1).

$${h}^{l + 1} , {x}^{l + 1} = f ( {h}^{l} , {x}^{l} ) \qquad ( 1 )$$

The superscript l represents a quantity of layers of the neural network. A 0^{th} layer of node features h⁰ are a set of type features of all nodes, and node coordinates x⁰ are a set of actual coordinates of all nodes in three-dimensional space during initialization. For example, specifically for each node in the graph data, for the graph data that uses a protein as aₙ input object and that is shown in FIG. 3,

$${h}_{i}^{0}$$

represents that a node t is an i^{th} amino acid, and may include four atom types (C, N, O, Cₐ), and

$${x}_{i}^{0}$$

represents actual coordinates of a node i in the three-dimensional space, that is, coordinates of four atoms in the i^{th} amino acid.

In some embodiments of the present disclosure, the determining a propagation message of the second node for the first node based on the vector of the node feature and the vector of the position feature includes: determining an equivariance feature vector between the first node and the second node based on vectors of the position features of the first node and the second node, the vectors being encoded by the previous hidden layer or perceptron in the EGNN model; and determining the propagation message of the second node for the first node based on the vectors of the node features of the first node and the second node in the graph data, the equivariance feature vector between the first node and the second node, and edge data between the first node and the second node, the vectors being encoded by the previous hidden layer or perceptron in the EGNN model.

For example, an operating principle of each hidden layer/perceptron may be represented by using the following formula (2) to formula (4).

$${m}_{i j} = {ϕ}_{m} ( {h}_{i}^{( l )} , {h}_{j}^{( l )} , \frac{{( {x}_{i j}^{( l )} )}^{⊤} {x}_{i j}^{( l )}}{{‖ {( {x}_{i j}^{l )} )}^{⊤} {x}_{i j}^{( l )} ‖}_{F}} , {e}_{i j} ) \qquad ( 2 )$$

φₘ is a function configured for calculating a propagation message mᵢⱼ between a node i and a node j, inputs of the function including a vector

$${h}_{i}^{( l )}$$

that is of a node feature of the node i and that is outputted by a hidden layer or perceptron at a l−1^{th} layer, a vector

$${h}_{j}^{( l )}$$

that is of a node feature of the node j and that is outputted by a hidden layer or perceptron at the l−1th layer (in an embodiment, the node i is adjacent to the node j), a equivariance feature vector

$$\frac{{( {x}_{i j}^{( l )} )}^{⊤} {x}_{i j}^{( l )}}{{‖ {( {x}_{i j}^{l )} )}^{⊤} {x}_{i j}^{( l )} ‖}_{F}}$$

between the node i and the node j, and edge data eᵢⱼ between the node i and the node j.

$${x}_{i j}^{( l )}$$

represents a displacement of the node j relative to the node i, and may be obtained through

$$( {x}_{j}^{( l )} - {x}_{i}^{( l )} ) .$$

A product of a transpose

$${( {x}_{i j}^{( l )} )}^{⊤} of {x}_{i j}^{( l )} and {x}_{i j}^{( l )}$$

may represent that rotation of the node j relative to the node i is eliminated (thereby ensuring rotation invariance). In other words,

$${( {x}_{i j}^{( l )} )}^{⊤} {x}_{i j}^{( l )}$$

actually represents a change degree of the node j relative to the node i caused by overall displacement and rotation. Then, a normalized equivariance feature vector may be obtained by dividing

$${( {x}_{i j}^{( l )} )}^{⊤} {x}_{i j}^{( l )} by {‖ {( {x}_{i j}^{l )} )}^{⊤} {x}_{i j}^{( l )} ‖}_{F} .$$

The hidden layer or perceptron obtains the propagation message mᵢⱼ by comprehensively calculating the foregoing four inputs. The propagation message mᵢⱼ is integrated with feature vectors of the node i and the node j, the edge data between the node i and the node j, and equivariance information of the node i and the node j.

Next, the hidden layer and/or perceptron calculates a vector of a node feature and a vector of a position feature of each node based on the propagation message.

In an embodiment, the first node may be adjacent to a plurality of second nodes, and the determining, based on the propagation message, a vector that is of a node feature and that is encoded by the hidden layer or perceptron includes: obtaining a plurality of propagation messages of the plurality of second nodes for the first node; and determining, based on the plurality of propagation messages and the vector that is of the node feature of the first node in the graph data and that is encoded by the previous hidden layer or perceptron in the EGNN model, the vector that is of the node feature of the first node and that is encoded by the hidden layer or perceptron. For example, the hidden layer and/or perceptron calculate/calculates the vector

$${h}_{i}^{( l + 1 )}$$

of the node feature of the node i based on the propagation message mᵢⱼ by using a formula (3). Certainly, the present disclosure is not limited thereto.

$${h}_{i}^{( l + 1 )} = {ϕ}_{h} ( {h}_{i}^{( l )} , {\sum }_{j ∈ 𝒩 ( i )} {m}_{i j} ) \qquad ( 3 )$$

φₕ is a function configured for calculating the vector

$${h}_{i}^{( l + 1 )}$$

of the node feature of the node i, inputs of the function including the vector

$${h}_{i}^{( l )}$$

that is of the node feature of the node i and that is outputted by a hidden layer or perceptron at the l−1^{th} layer, and a propagation message mᵢⱼ of all nodes j∈ [symbol] (i) adjacent to the node i for the node i. In this way, the vector

$${h}_{i}^{( l + 1 )}$$

that is of the node feature of the node i and that is outputted by a hidden layer or perceptron at a l^{th} layer may be obtained.

In an embodiment, the first node may be adjacent to a plurality of second nodes, and the determining, based on the propagation message, a vector that is of a position feature and that is encoded by the hidden layer or perceptron includes: obtaining the plurality of propagation messages of the plurality of second nodes for the first node; and determining, based on the plurality of propagation messages and vectors of position features of the first node and the plurality of second nodes in the graph data, the vector that is of the position feature of the first node and that is encoded by the hidden layer or perceptron, the vectors being encoded by the previous hidden layer or perceptron in the EGNN model. For example, the hidden layer and/or perceptron calculates the vector

$${x}_{i}^{( l + 1 )}$$

of the node feature of the node i based on the propagation message by using a formula (4). Certainly, the present disclosure is not limited thereto.

$${x}_{i}^{( l + 1 )} = {x}_{i}^{( l )} + \frac{1}{\vert 𝒩 ( i ) \vert} {\sum }_{j ∈ 𝒩 ( i )} {x}_{ij}^{( l )} {ϕ}_{x} ( {m}_{ij} ) \qquad ( 4 )$$

The formula (4) is a function configured for calculating a vector

$${x}_{i}^{( l + 1 )}$$

of a position feature of the node i, inputs of the formula (4) including a vector

$${x}_{i}^{( l + 1 )}$$

that is of a position feature of the node i and that is outputted by a hidden layer or perceptron at the l−1^{th} layer, and the propagation message mᵢⱼ of all nodes j∈ [symbol] (i) adjacent to the node i for the node i. φₓ is a function configured for mapping the propagation message mᵢⱼ of the node j for the node i to position information. Through

$$\frac{1}{\vert 𝒩 ( i ) \vert} {\sum }_{j ∈ 𝒩 ( i )} {x}_{ij}^{( l )} {ϕ}_{x} ( {m}_{ij} ) , {x}_{i}^{( l + 1 )}$$

is integrated with position information

$${x}_{i}^{( l )}$$

of

$${x}_{i}^{( l + 1 )}$$

and position information corresponding to a neighboring node

$${x}_{j}^{( l )} .$$

In this way, the vector

$${x}_{i}^{( l + 1 )}$$

that is of the position feature of the node i and that is outputted by the hidden layer or perceptron at the l^{th} layer of may be obtained.

The vector h∈R^{d} of the node feature and the vector x∈R³ of the position feature of each node (for example, each amino acid) are obtained by using the foregoing described EGNN model. In an implementation, a hidden representation Z of the node may be obtained by combining the vectors, that is, Z=(H,X)=φ(G)∈R^{d}. Then, the d-dimensional hidden representation Z is discretized (clustered), to obtain a representation c∈R^{d} of a cluster center thereof. Alternatively, the vector h∈R^{d} of the node feature or the vector x∈R³ of the position feature may be directly discretized (clustered), to obtain a representation c∈R^{d} or c∈R³ of a cluster center thereof. In this case, the vector h∈R^{d} of the node feature or the vector x∈R³ of the position feature may alternatively be directly used as a hidden representation zᵢ.

For example, a hidden representation zᵢ may be outputted for each node i. A cluster center cᵢ nearest to the node vector zᵢ is found by using the discretizer. It is assumed that a number of the cluster center cᵢ is indᵢ. In this case, the entire graph data may be encoded into a data sequence S={ind₀, ind₁, . . . , indₙ)}, and indᵢ∈Z⁺. Specifically, for the graph data that uses the protein or macromolecule as the input object, each amino acid may output a hidden representation zᵢ, and then find the cluster center cᵢ nearest to zᵢ. It is assumed that a number is indᵢ. In this case, an entire protein chain may be encoded into a data sequence S={ind₀, ind₁, . . . , indₙ)}. indᵢ∈Z⁺, and a range is [1, 20].

Therefore, the data sequence S obtained by using the graph encoder in FIG. 5 can represent, with high precision, a three-dimensional space structure and a relationship feature between nodes of the graph data without occupying excessively large storage space. In addition, the data sequence S is also easy to perform retrieval in the subsequent operation S303. Specifically, for a protein retrieval tool, in this embodiment of the present disclosure, the graph data is converted into the data sequence, to convert a structure comparison task of the protein into a sequence comparison task. The data sequence obtained through conversion in operation S302 ensures structure information comparison, and improves a retrieval speed through sequence comparison.

A person skilled in the art is to understand that the present disclosure is not limited thereto. In addition to retrieving the data sequence by using the foregoing clustering-based method, a maximum probability decoding solution may alternatively be used. For example, a probability distribution of the graph encoder may be searched for better matching code to improve a retrieval speed of a similar data sequence.

Next, operation S303 is further described with reference to FIG. 6. FIG. 6 is a diagram of operation S303 according to an embodiment of the present disclosure.

In an embodiment, operation S303 includes: Compare the data sequence with another data sequence in the graph database by using a dynamic programming algorithm, to obtain a comparison result indicating a local similarity between the data sequence and the another data sequence in the graph database; and determine the output object corresponding to the input object based on the comparison result. Certainly, the present disclosure is not limited thereto.

As shown in FIG. 6, the data sequence S obtained in operation S302 may be compared with another data sequence in the graph database by using a Smith-Waterman algorithm (which is an example of the dynamic programming algorithm), to quickly find a data sequence S′ closest to the data sequence S in operation S303, and index the output object closest to the input object based on the data sequence S′.

The Smith-Waterman algorithm is an algorithm configured for comparing two data sequences, and is configured for searching for a local similarity between two data sequences based on a dynamic programming principle, to search for an optimal alignment manner and find a sub-data sequence having a highest similarity.

A main process of using the Smith-Waterman algorithm may be briefly described as follows. First, a matrix is created to store a comparison score between two data sequences. A dimension of the matrix is related to lengths of two input data sequences. In this embodiment of the present disclosure, the dimension may be taken as [1, 20]. Usually, an initial row and column of the matrix are initialized to be 0. Next, similarity scores of elements of two data sequences are calculated row by row and column by column starting from the 1^{st} element at an upper left corner of the matrix. The similarity score includes at least one of a matching score, a mismatch penalty, and a deletion penalty, or a weighted sum of the three items, or a maximum value of the three items. In a process of score calculation, an element having a maximum score and a position of the element having the maximum score are always recorded. Then, backward backtracking is performed according to a score rule (matching and mismatching) starting from the position of the element having the maximum score, to find an optimal comparison path, that is, optimal local alignment of the two data sequences. The matching score, the mismatch penalty, and the deletion penalty may be defined by a user.

The Smith-Waterman algorithm may be configured for searching for a similarity in a local range without global alignment. Therefore, two word fragments closest to each other in the two data sequences may be found. This is particularly useful when the data sequences are used as protein sequences for comparison, which can find protein structure fragments or amino acid fragments having similar functional areas.

In addition, in this embodiment of the present disclosure, a more precise and efficient retrieval method that may be developed in the future may further be used, to improve retrieval accuracy and efficiency.

Next, training of a graph encoder is further described with reference to FIG. 7. FIG. 7 is a diagram of training a graph encoder according to an embodiment of the present disclosure.

In an example, training data configured for training an unlocking model in this embodiment of the present disclosure may be from a protein data bank (PDB) database, and the database stores data of a protein of a known structure and a known function. In some cases, to extend the training data, an AlphaFold2 model may also be used to predict some protein structures and increase training data of the model.

To train the graph encoder in the retrieval model according to this embodiment of the present disclosure, so as to enable the graph encoder to capture important information and features in graph data, a graph decoder shown in FIG. 7 may be used. The graph decoder is a neural network model that can restore an output vector H,X of the graph encoder to the graph data.

To enable the graph encoder to learn of node type information of the input graph data, in an embodiment, the training of the graph encoder includes: determining, by using the graph encoder during training, a predicted type value of each node based on node data corresponding to each node in the graph data; determining a type prediction loss based on the predicted type value of each node and an actual type value of each node; and adjusting a neural network parameter of the graph encoder based on the type prediction loss. For example, the graph encoder may be trained by using a type prediction loss Lₐₐ shown in the following formula (5).

$${L}_{aa} = {\sum }_{i = 1}^{n} C ( ϕ ( {h}_{i} ) , {aa}_{i} ) \qquad ( 5 )$$

Inputs of the type prediction loss Lₐₐ include φ(hᵢ) and aaᵢ. φ(hᵢ) is a type of a node predicted based on a node feature hᵢ outputted by the graph encoder. Specifically, for graph data that uses a protein as an input project, φ(hᵢ) is a type of an amino acid predicted based on the graph encoder, and aaᵢ is an actual type of the amino acid. C(φ(hᵢ),aaᵢ) represents a cross entropy loss of φ(hᵢ) and aaᵢ. In this way, the type prediction loss represents a type prediction loss of all amino acids in a protein. In an embodiment, in the field of life science data analysis, the type prediction loss is also referred to as an amino acid prediction loss.

To enable the graph encoder and the graph decoder to learn of three-dimensional structure information of the input graph data, the training of the graph encoder may be assisted by using the graph decoder. In an embodiment, the training of the graph encoder includes: determining, by using the graph encoder during training, a predicted hidden representation of each node based on the node data corresponding to each node in the graph data; determining restored position information of each node based on the predicted hidden representation of each node by using the graph decoder during training; determining a position mismatch loss based on the position information of each node in three-dimensional space and the restored position information of each node; and adjusting a neural network parameter of the graph encoder and/or a neural network parameter of the graph decoder based on the position mismatch loss.

For example, the graph encoder and the graph decoder may be trained by using a position mismatch loss L_{coor} shown in the following formula (6).

$${L}_{coor} = kabsch ( {X}^{′} , X ) \qquad ( 6 )$$

The position mismatch loss L_{coor} includes X′ and X. X′ is a set of coordinates of nodes restored by the graph decoder, and X is a set of coordinates of nodes of the graph data inputted into the graph encoder. Operations such as rotation and translation are performed on the coordinate X′ and the coordinate X by using kabsch(X′,X) in space, so that the two coordinates are aligned. kabsch(X′,X) is a minimum error after alignment. In an embodiment, in the field of life science data analysis, the position mismatch loss is also referred to as a coordinate alignment loss.

The type prediction loss Lₐₐ and the position mismatch loss L_{coor} may be combined by using a formula (7), so that in a training process, the graph encoder and the graph decoder are simultaneously trained.

$$L = {L}_{aa} + {L}_{coor} \qquad ( 7 )$$

A process of training the graph encoder and the graph decoder simultaneously includes at least a process of adjusting parameter values of neurons in the graph encoder and the graph decoder to minimize a loss L.

The graph decoder according to this embodiment of the present disclosure is used only in a training stage, and does not affect inferring efficiency of the graph encoder. When a sample quantity is limited, robustness of learning of a graph representation can be improved by using the loss L, because the loss L enables the graph encoder to pay more attention to global information and a topology structure in a graph, rather than local noise or irrelevant features. In addition, the loss L can also reduce an overfitting risk because the loss L can prevent the graph encoder from overfitting a specific sample or sub-graph in the training data and ignoring a generalization capability.

In addition to training the graph encoder and the decoder in the retrieval model by using the loss L, a discretization loss L_{disc}=∥c−z∥² may also be considered in the training process to train the graph encoder. The discretization loss L_{disc} indicates a loss caused by vectorization of a hidden representation z of the node, and is a difference between a cluster center c and a sequence z obtained through discretization. A weighted sum of the loss L and the discretization loss L_{disc} may be used as a loss function of an entire model, to comprehensively consider losses, and improve performance of the graph encoder or the graph decoder.

The foregoing losses can significantly improve performance of the retrieval model. Specifically, for graph data corresponding to a protein or a macromolecule, the type prediction loss Lₐₐ helps to improve type prediction accuracy of an amino acid sequence, and the discretization loss L_{disc} helps improve structure prediction accuracy of the amino acid sequence. According to the training method in this embodiment of the present disclosure, the graph encoder and the graph decoder can be trained more efficiently, so that the performance of the graph encoder and the graph decoder is higher, and structure information in the graph data can be learned better.

An embodiment of the present disclosure further provides a data retrieval apparatus, configured to retrieve an input object in a graph database. The apparatus includes: a graph data constructing module, configured to obtain graph data corresponding to the input object, the graph data including a plurality of nodes and node data corresponding to each node, and the node data including position information of the node in three-dimensional pace; an encoding module, configured to encode and discretize the graph data by using a graph encoder, to obtain a data sequence corresponding to the input object, the data sequence representing discretized information of at least one of node features or position features of the plurality of nodes, and the discretized information being obtained through encoding; and a comparison module, configured to: retrieve, in the graph database, a target data sequence satisfying a similarity threshold with the data sequence, and determine an output object corresponding to the input object based on the retrieved target data sequence.

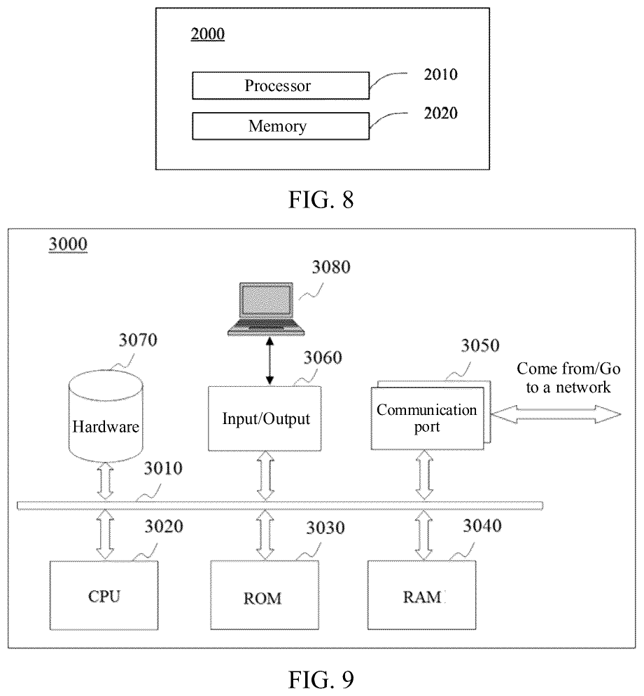

According to another aspect of the present disclosure, an electronic device is further provided, and is configured to implement the method according to the embodiments of the present disclosure. FIG. 8 is a diagram of an electronic device 2000 according to an embodiment of the present disclosure.

As shown in FIG. 8, the electronic device 2000 may include one or more processors 2010 and one or more memories 2020. The memory 2020 stores computer-readable code. When the computer-readable code is executed by the one or more processors 2010, the foregoing method may be performed.

The processor in this embodiment of the present disclosure may be an integrated circuit chip having a signal processing capability. The processor may be a general-purpose processor, a digital signal processor (DSP), an application-specific integrated circuit (ASIC), a field programmable gate array (FPGA) or another programmable logic device, a discrete gate or a transistor logic device, or a discrete hardware component. The processor may implement or perform the methods, the operations, and logic block diagrams that are disclosed in the embodiments of the present disclosure. The general-purpose processor may be a microprocessor, or the processor may be any conventional processor or the like, and may be an X86 architecture or an ARM architecture.

Generally, various example embodiments of the present disclosure may be implemented in hardware or a dedicated circuit, software, firmware, logic, or any combination thereof. Some aspects may be implemented in hardware, and other aspects may be implemented in firmware or software that may be executed by a controller, a microprocessor, or another computing device. When aspects of the embodiments of the present disclosure are shown or described as block diagrams or flowcharts, or represented by using some other figures, the blocks, apparatuses, systems, techniques, or methods described herein may be implemented in hardware, software, firmware, a dedicated circuit or logic, general-purpose hardware or a controller or another computing device, or some combination thereof as non-restrictive examples.

For example, the methods or apparatuses according to the embodiments of the present disclosure may alternatively be implemented with the help of an architecture of a computing device 3000 shown in FIG. 9. As shown in FIG. 9, the computing device 3000 may include a bus 3010, one or more CPUs 3020, a read-only memory (ROM) 3030, a random access memory (RAM) 3040, a communication port 3050 connected to a network, an input/output component 3060, a hard disk 3070, and the like. A storage device, for example, the ROM 3030 or the hard disk 3070, in the computing device 3000 may store various data or files used for processing and/or communication in the method provided in the present disclosure and program instructions executed by the CPU. The computing device 3000 may further include a user interface 3080. Certainly, the architecture shown in FIG. 7 is merely an example. When different devices are implemented, based on an actual requirement, one or more components in the computing device shown in FIG. 7 may be omitted.

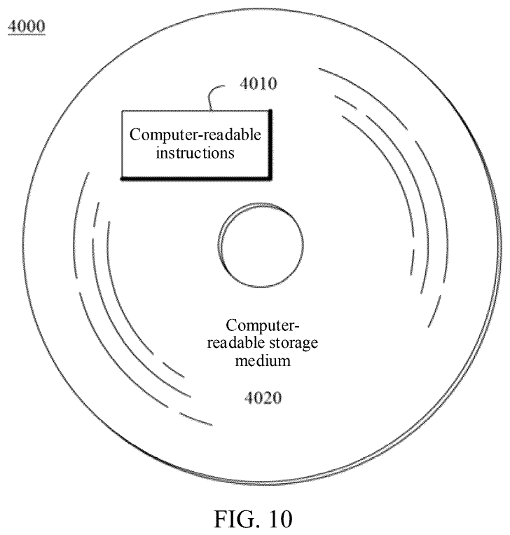

According to still another aspect of the present disclosure, a computer-readable storage medium is further provided. FIG. 10 is a diagram of a storage medium 4000 according to the present disclosure.

As shown in FIG. 10, the computer storage medium 4020 stores computer-readable instructions 4010. The computer-readable instructions 4010, when executed by a processor, may perform the method according to the embodiments of the present disclosure described with reference to the foregoing accompanying drawings. The computer-readable storage medium in this embodiment of the present disclosure may be a volatile memory or a non-volatile memory, or may include both a volatile memory and a non-volatile memory. The non-volatile memory may be a read-only memory (ROM), a programmable read-only memory (PROM), an erasable programmable read-only memory (EPROM), an electrically erasable programmable read-only memory (EEPROM), or a flash memory. The volatile memory may be a random access memory (RAM), and is used as an external cache. Through examples but not restrictive descriptions, RAMs in many forms are usable, such as a static random access memory (SRAM), a dynamic random access memory (DRAM), a synchronous dynamic random access memory (SDRAM), a double data rate synchronous dynamic random access memory (DDRSDRAM), an enhanced synchronous dynamic random access memory (ESDRAM), a synchlink dynamic random access memory (SLDRAM), and a direct rambus random access memory (DR RAM). The memories in the method described in this specification are intended to include, but are not limited to these and any other suitable types of memories. The memories in the method described in this specification are intended to include, but are not limited to these and any other suitable types of memories.

An embodiment of the present disclosure further provides a computer program product or a computer program. The computer program product or the computer program includes computer instructions, the computer instructions being stored in a computer-readable storage medium. A processor of a computer device reads the computer instructions from the computer-readable storage medium. The processor executes the computer instructions, to enable the computer device to perform the method according to the embodiments of the present disclosure.

Flowcharts and block diagrams in the accompanying drawings illustrate architectures, functions, and operations that may be implemented using the system, the method, and the computer program product according to various embodiments of the present disclosure. In this regard, each box in the flowchart or the block diagram may represent a module, a program segment, or a part of code. The module, the program segment, or the part of code includes one or more executable instructions used for implementing designated logic functions. In some implementations used as substitutes, functions annotated in boxes may alternatively occur in a sequence different from that annotated in the accompanying drawings. For example, actually two boxes shown in succession may be performed basically in parallel, or sometimes the two boxes may be performed in a reverse sequence. This is determined by a related function. Each box in a block diagram and/or a flowchart and a combination of boxes in the block diagram and/or the flowchart may be implemented by using a dedicated hardware-based system for performing a specified function or operation, or may be implemented by using a combination of dedicated hardware and computer instructions.

Generally, various example embodiments of the present disclosure may be implemented in hardware or a dedicated circuit, software, firmware, logic, or any combination thereof. Some aspects may be implemented in hardware, and other aspects may be implemented in firmware or software that may be executed by a controller, a microprocessor, or another computing device. When aspects of the embodiments of the present disclosure are shown or described as block diagrams or flowcharts, or represented by using some other figures, the blocks, apparatuses, systems, techniques, or methods described herein may be implemented in hardware, software, firmware, a dedicated circuit or logic, general-purpose hardware or a controller or another computing device, or some combination thereof as non-restrictive examples.

The foregoing example embodiments of the present disclosure described in detail are merely illustrative but are not restrictive. A person skilled in the art may understand that various modifications and combinations may be made to the embodiments or features thereof without departing from the principle and spirit of the present disclosure.

What is claimed is:

1. A retrieval method, comprising:
obtaining graph data corresponding to an input object, the graph data comprising a plurality of nodes and node data corresponding to each node;
encoding and discretizing the graph data by using a graph encoder, to obtain a data sequence corresponding to the input object, the data sequence representing discretized information of at least one of node features or position features of the plurality of nodes, and the discretized information being obtained through the encoding; and
retrieving, in a graph database, a target data sequence satisfying a similarity threshold with the data sequence, and determining an output object corresponding to the input object based on the retrieved target data sequence.

2. The retrieval method according to claim 1, wherein the input object has a plurality of internal components distributed in three dimensions in a three-dimensional space, each node in the graph data corresponding to the input object corresponds to one or more internal components in the input object, and the node data corresponding to each node comprises position information of the node in the three-dimensional space.

3. The retrieval method according to claim 2, wherein the graph data further comprises edge data connected between nodes, the edge data indicating an association relationship between two internal components in the input object.

4. The retrieval method according to claim 1, wherein the graph data corresponding to the input object satisfies at least one of rotation equivariance, translation equivariance, or scaling equivariance.

5. The retrieval method according to claim 1, wherein the input object is a protein, and the obtaining graph data corresponding to an input object comprises:
determining each node and the node data of the node in the graph data corresponding to the input object based on sequence information of a plurality of amino acids comprised in the protein, each node corresponding to one amino acid, the node data corresponding to a node comprising type information of atoms on a main chain of the amino acid and coordinates of the atoms in the three-dimensional space; and
determining each piece of edge data in the graph data based on the node data of each node in the graph data.

6. The retrieval method according to claim 5, wherein the determining each piece of edge data in the graph data based on the node data of each node in the graph data comprises:
creating and storing, based on a chemical bond between atoms of two amino acids, edge data corresponding to the chemical bond; or
creating and storing, in response to that a distance between atoms of two amino acids is less than a predetermined threshold, edge data for connecting the atoms of the two amino acids.

7. The retrieval method according to claim 1, wherein the graph encoder comprises an equivariant graph neural network model and a discretizer, and the encoding and discretizing the graph data by using a graph encoder comprises:
encoding the graph data by using the equivariant graph neural network model, to obtain the node feature and the position feature of each node in the graph data; and
performing vector quantization on at least one of the node feature or the position feature of each node by using the discretizer, to obtain the data sequence representing the discretized information of at least one of the node features or the position features of the plurality of nodes.

8. The method according to claim 7, wherein the equivariant graph neural network model comprises a plurality of hidden layers or perceptrons, each hidden layer or perceptron encodes a vector of the node feature and a vector of a spatial position of each node, and encoding of each hidden layer or perceptron other than the 1^{st} hidden layer or perceptron comprises:
obtaining vectors of node features and vectors of position features of a first node and a second node in the graph data, the vectors being encoded by a previous hidden layer or perceptron in the equivariant graph neural network model; and
determining a propagation message of the second node for the first node based on the vectors of the node feature and the vectors of the position feature of the first and the second nodes; and
determining, based on the propagation message, a vector of a node feature and a vector of a position feature that are encoded by the hidden layer or perceptron.

9. The method according to claim 8, wherein the determining a propagation message of the second node for the first node based on the vectors of the node feature and the vectors of the position feature of the first and the second nodes comprises:
determining an equivariance feature vector between the first node and the second node based on the vectors of the position features of the first node and the second node, the vectors being encoded by the previous hidden layer or perceptron in the equivariant graph neural network model; and
determining the propagation message of the second node for the first node based on the vectors of the node features of the first node and the second node in the graph data, the equivariance feature vector between the first node and the second node, and edge data between the first node and the second node, the vectors being encoded by the previous hidden layer or perceptron in the equivariant graph neural network model.

10. The method according to claim 9, wherein the first node is adjacent to a plurality of second nodes, and the determining, based on the propagation message, a vector that is of a node feature and that is encoded by the hidden layer or perceptron comprises:
obtaining a plurality of propagation messages of the plurality of second nodes for the first node; and
determining, based on the plurality of propagation messages and the vector that is of the node feature of the first node in the graph data and that is encoded by the previous hidden layer or perceptron in the equivariant graph neural network model, the vector that is of the node feature of the first node and that is encoded by the hidden layer or perceptron.

11. The method according to claim 8, wherein the first node is adjacent to the plurality of second nodes, and the determining, based on the propagation message, a vector that is of a position feature and that is encoded by the hidden layer or perceptron comprises:
obtaining the plurality of propagation messages of the plurality of second nodes for the first node; and
determining, based on the plurality of propagation messages and vectors of position features of the first node and the plurality of second nodes in the graph data, the vector that is of the position feature of the first node and that is encoded by the hidden layer or perceptron, the vectors being encoded by the previous hidden layer or perceptron in the equivariant graph neural network model.

12. The method according to claim 1, wherein the retrieving, in a graph database, a target data sequence satisfying a similarity threshold with the data sequence comprises:
comparing the data sequence with a candidate data sequence in the graph database by using a dynamic programming algorithm, to obtain a comparison result indicating a local similarity between the data sequence and the candidate data sequence in the graph database; and
determining the output object corresponding to the input object based on the comparison result.

13. The method according to claim 1, wherein training of the graph encoder comprises:
determining, by using the graph encoder during training, a predicted type value of each node based on the node data corresponding to each node in the graph data;
determining a type prediction loss based on the predicted type value of each node and an actual type value of each node; and
adjusting a neural network parameter of the graph encoder based on the type prediction loss.

14. The method according to claim 13, wherein the training of the graph encoder is assisted by a graph decoder and comprises:
determining, by using the graph encoder during training, a predicted hidden representation of each node based on the node data corresponding to each node in the graph data;
determining restored position information of each node based on the predicted hidden representation of each node by using the graph decoder during training;
determining a position mismatch loss based on the position information of each node in the three-dimensional space and the restored position information of each node; and
adjusting a neural network parameter of the graph encoder and/or a neural network parameter of the graph decoder based on the position mismatch loss.

15. A data retrieval apparatus, comprising:
one or more processors; and
one or more memories, the memory having a computer-executable program stored therein, and the processor, when executing the computer-executable program, being configured to perform:
obtaining graph data corresponding to an input object, the graph data comprising a plurality of nodes and node data corresponding to each node;
encoding and discretizing the graph data by using a graph encoder, to obtain a data sequence corresponding to the input object, the data sequence representing discretized information of at least one of node features or position features of the plurality of nodes, and the discretized information being obtained through the encoding; and
retrieving, in a graph database, a target data sequence satisfying a similarity threshold with the data sequence, and determining an output object corresponding to the input object based on the retrieved target data sequence.

16. The retrieval apparatus according to claim 15, wherein the input object has a plurality of internal components distributed in three dimensions in a three-dimensional space, each node in the graph data corresponding to the input object corresponds to one or more internal components in the input object, and the node data corresponding to each node comprises position information of the node in the three-dimensional space.

17. The retrieval apparatus according to claim 16, wherein the graph data further comprises edge data connected between nodes, the edge data indicating an association relationship between two internal components in the input object.

18. The retrieval apparatus according to claim 15, wherein the graph data corresponding to the input object satisfies at least one of rotation equivariance, translation equivariance, or scaling equivariance.

19. The retrieval apparatus according to claim 15, wherein the input object is a protein, and the obtaining graph data corresponding to an input object comprises:
determining each node and the node data of the node in the graph data corresponding to the input object based on sequence information of a plurality of amino acids comprised in the protein, each node corresponding to one amino acid, the node data corresponding to a node comprising type information of atoms on a main chain of the amino acid and coordinates of the atoms in the three-dimensional space; and
determining each piece of edge data in the graph data based on the node data of each node in the graph data.

20. A non-transitory computer-readable storage medium, having computer instructions stored therein, when the computer instructions are executed by at least one processor, causing the at least one processor to perform:
obtaining graph data corresponding to an input object, the graph data comprising a plurality of nodes and node data corresponding to each node;
encoding and discretizing the graph data by using a graph encoder, to obtain a data sequence corresponding to the input object, the data sequence representing discretized information of at least one of node features or position features of the plurality of nodes, and the discretized information being obtained through the encoding; and
retrieving, in a graph database, a target data sequence satisfying a similarity threshold with the data sequence, and determining an output object corresponding to the input object based on the retrieved target data sequence.
